(19) United States
(12) Patent Application Publication
(10) Pub. No.: US 2024/0164977 A1
(43) Pub. Date: May 23, 2024

(54) Grounding Skin Scraper

(71) Applicant: Jeremy Madvin, Westlake Village, CA (US)

(72) Inventor: Jeremy Madvin, Westlake Village, CA (US)

(21) Appl. No.: 17/990,689

(22) Filed: Nov. 20, 2022

(51) Int. Cl. A61H 7/00

(52) U.S. Cl. CPC A61H 7/003; A61H 2201/10

(57) ABSTRACT

The present invention relates to a handheld massaging, scrapping, and topical enhancer tool which allows the user to scrape their skin with the added benefit of grounding. The assembly is easy to use, portable and can be used with or without external medicated or non-medicated liquids/creams.

### BACKGROUND

### Field of the Invention

The present invention relates to a grounding apparatus or set thereof or method for use in taking care of skin. The invention is related to a grounding skincare tool which, in addition to the basic function of massaging, scraping, and assisting in the application of topical creams, oils, and liniments, also helps to target the exit of excess energy from the face or body and the entry of ions from the earth directly to the area in contact with the skin scraper.

### Description of the Related Art

Multi-disciplinary research has revealed that electrically conductive contact of the human body with the surface of the Earth (grounding or earthing) produces intriguing effects on physiology and health. Such results relate to inflammation, immune responses, wound healing, and prevention and treatment of chronic inflammatory and autoimmune diseases.

Grounding or earthing refers to direct skin contact with the surface of the Earth, such as with bare feet or hands or with various grounding systems. Subjective reports that walking barefoot on the Earth enhances health and provides feelings of well-being can be found in the literature and practices of diverse cultures from around the world. For various reasons, many individuals are reluctant to walk outside barefoot unless they are on holiday at the beach. Experience and measurements show that sustained contact with the Earth yields sustained benefits. Various grounding systems are available that enable frequent contact with the Earth, such as while sleeping, sitting at a computer, or walking outdoors. These are simple conductive systems in the form of sheets, mats, wrist or ankle bands, adhesive patches that can be used inside the home or office, and footwear. These applications are connected to the Earth via a cord inserted into a grounded wall outlet or attached to a ground rod placed in the soil outside below a window. For footwear applications, a conductive plug is positioned in the shoe sole at the ball of the foot, under the metatarsals, at the acupuncture point known as the Kidney. From a practical standpoint, these methods offer a convenient and routine, user-friendly approach to grounding or earthing. They can also be used in clinical situations, as will be described in the section entitled Summary of findings to date.

Grounding reduces or even prevents the cardinal signs of inflammation following injury: redness, heat, swelling, pain, and loss of function. In a small study on grounding and heart health, ten healthy participants were grounded using patches on the palms of their hands and soles of their feet.

Blood measurements were taken before and after grounding to determine any changes in red blood cell fluidity, which plays a role in heart health. The results indicated significantly less red blood cell clumping after grounding, which suggests benefits for cardiovascular health.

Another slightly larger study examined the role of grounding on post-exercise muscle damage. Researchers used both grounding patches and mats and measured creatine kinase, white blood cell count, and pain levels before and after grounding.

Blood work indicated that grounding reduced muscle damage and pain in participants. This suggests that grounding may influence healing abilities. By looking at prior art, multiple advances have been seen in grounding equipment to provide better tools to users. For instance, a U.S. Pat. No. 6,683,779B2 discusses personal body grounding system for collecting and removing unnatural electrical charges from a human body, includes a grounding pad having a layer of carbon fibers and a conductor substantially extending across the layer in conductive contact with the carbon fibers. A ground lead is conductively coupled to the grounding pad conductor at one end thereof and conductively coupled to a grounded anchor at a second end thereof. Preferably, the grounded anchor is placed directly into the earth, although in certain applications constitutes a metal grounding component. The system may include a wall plate in conductive connection with the grounded anchor. The ground lead is removably attached to the wall plate via a connector attached thereto. The grounding pad may be configured as a sleeping pad, seat pad, strap, or patch attachable to the human body.

A GB patent 8,864,64A on grounding devices discusses the human body is discharged of static electricity while in bed using bedding or pillow slips 12, 14 made of a fabric incorporating threads of bare copper, steel, platinum, or other metal connected to ground, e.g., the “earth” terminal of a three-pin socket 40. A suitable adaptor 22 is described.

A U.S. Pat. No. 4,577,256A on woven stretchable grounding strap, which may be comfortably worn on the wrist to drain or wick static electrical charges from the wearer. Conductive fibers on the inside surface of the strap contact the skin and conduct electrical charges to a grounding cord attached to the strap. Face yarns exposed on the outer surface may be woven to form designs. The woven fabric material of the strap is attached to a clasp allowing the strap to be adjustable in size. Because of the woven nature of the fabric material and the adjustable clasp, the strap is more comfortable than other conductive elastic wrist straps. The woven fabric material is particularly advantageous because it stretches easily, it is inexpensive, and it does not roll over onto itself as it is being drawn over the hand.

A WO patent 2,008,033945A2 discusses personal body grounding systems to reduce and prevent inflammation. The invention discloses methods and systems for conductively coupling the body and, more specifically, the bare feet of an animal or human body to the earth in situations where conductive contact with the earth is otherwise impossible, such as inside a home or other building or in a bed. Various embodiments of the invention allow earth's mobile surface charge of free electrons to flow from the earth to the body (similar to that which occurs naturally when an animal or human is standing barefoot on the earth) for the purpose of neutralizing excess or residual immune system-produced reactive oxygen species free radicals that cause physiological deficiencies and disturbances in the body such as acute or chronic inflammation.

Multiple advancements have been presented in the prior art. However, these solutions are limited and restricted to their conventional systems. The current invention aims to disclose a device that provides the skin scraper function along with the grounding function, which enables the user to have additional benefits as compared to similar skin care devices available in the market.

None of the previous inventions and patents, taken either singly or in combination, is seen to describe the instant invention as claimed. Hence, the inventor of the present invention proposes to resolve and surmount existent technical difficulties to eliminate the aforementioned shortcomings of prior art.

### SUMMARY

In light of the disadvantages of the prior art, the following summary is provided to facilitate an understanding of some of the innovative features unique to the present invention and is not intended to be a full description. A full appreciation of the various aspects of the invention can be gained by taking the entire specification, claims, drawings, and abstract as a whole.

The primary desirable object of the present invention is to provide a novel and improved form of the simplest and most efficient two-in-one skincare scraper device with a grounding function.

It is also the invention's primary objective to provide a hand-held assembly that allows the user to gently scrape the skin.

It is another objective of the invention to provide a handheld assembly that can act, including but not limited to a facial massage tool, facial care tools, oil, liniment or cream applicator, cosmetic spatulas, lifting & exfoliating tool, Microdermabrasion device, scraping knife, dead skin grinding scraper, or full body exfoliator.

Another object of the present invention is to provide a skincare tool that provides a grounding function that helps to target the exit of excess energy from the face or body and the entry of ions from the earth directly to the area in contact with the skin scraper.

A further object of the present invention is to provide an assembly that is easy to use, hold, and clean.

It is also the objective of the invention to provide a smart assembly that provides ease of use and convenience to the user and provides multiple functionalities to the user.

It is also the objective of the invention to provide an efficient and easy assembly that is cost-effective and convenient

Thus, it is the objective to provide advancement in the skin care industry with earthing functionality. Other aspects, advantages, and novel features of the present invention will become apparent from the detailed description of the invention when considered in conjunction with the accompanying drawings.

This Summary is provided merely for purposes of summarizing some example embodiments so as to provide a basic understanding of some aspects of the subject matter described herein. Accordingly, it will be appreciated that the above-described features are merely examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF DRAWINGS

The accompanying figures, where like reference numerals refer to identical or functionally similar elements throughout the separate views, together with the detailed description below, are incorporated in and form part of the specification and serve to further illustrate embodiments of concepts that include the claimed invention, and explain various principles and advantages of those embodiments.

FIG. 1 shows an installed view of the claimed assembly as per preferred embodiments of the invention.

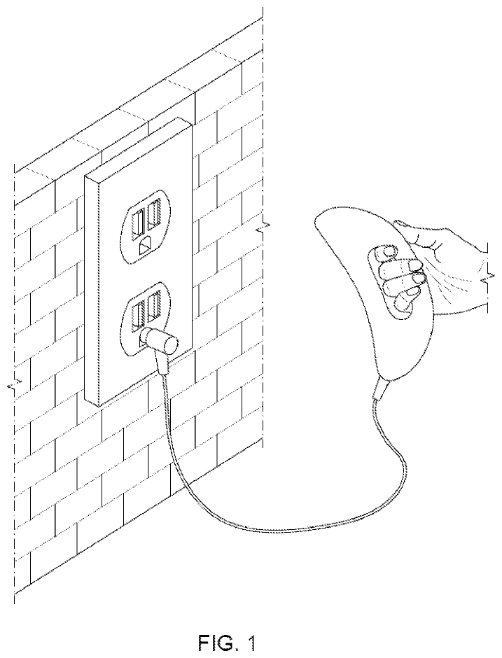

FIG. 2 shows the installed view of the claimed assembly with a laptop as per preferred embodiments of the invention.

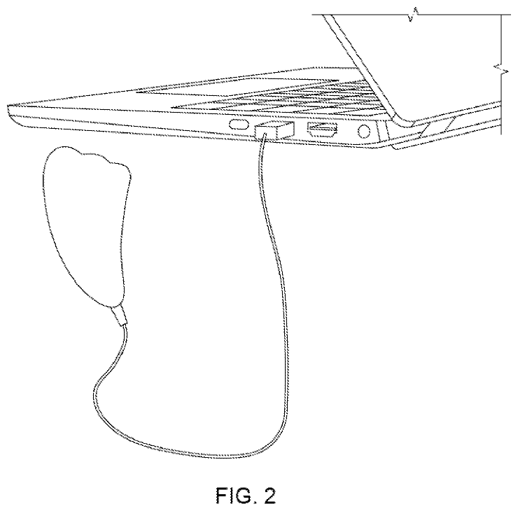

FIG. 3 shows another installed view of the claimed assembly with a laptop as per preferred embodiments of the invention.

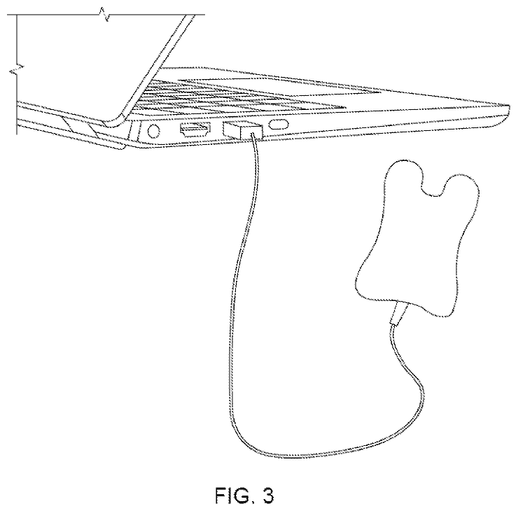

FIG. 4 shows the ground-installed view of the claimed assembly as per preferred embodiments of the invention.

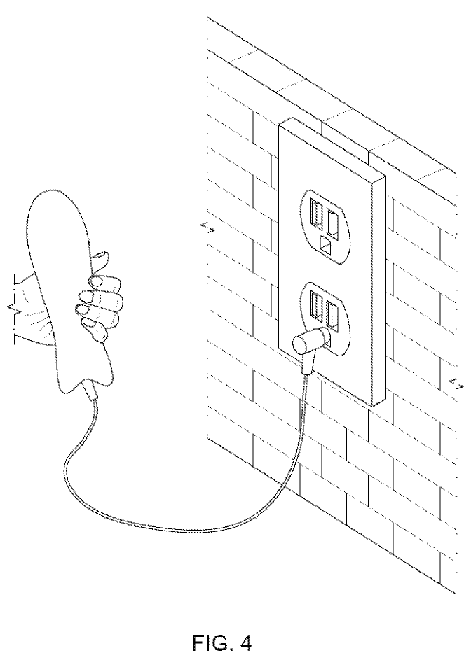

FIG. 5 shows another installed view of the claimed assembly as per preferred embodiments of the invention.

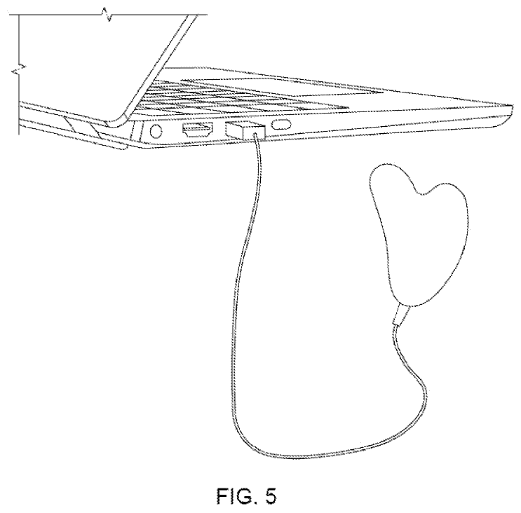

FIG. 6 shows the use of claimed assembly as per preferred embodiments of the invention.

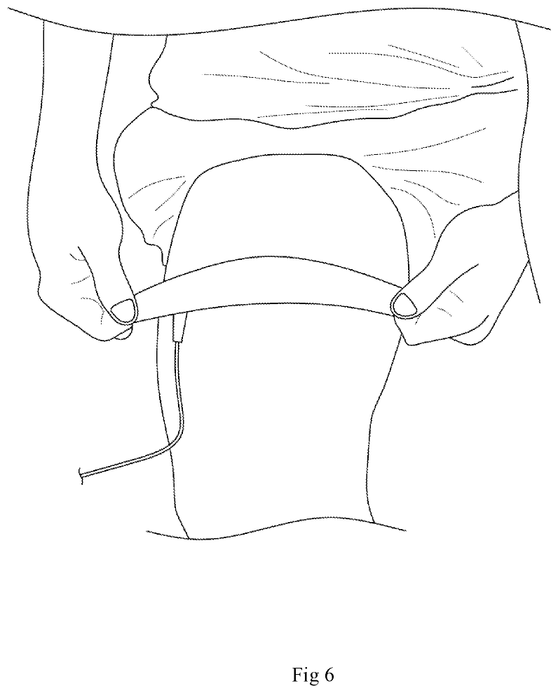

FIG. 7 shows the perspective use of the claimed assembly as per preferred embodiments of the invention.

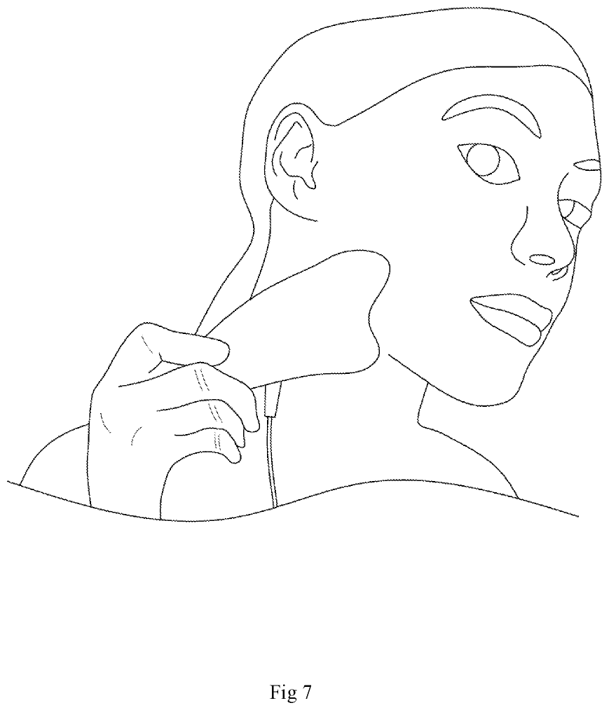

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

The apparatus and method components have been represented where appropriate by conventional symbols in the drawings, showing only those specific details that are pertinent to understanding the embodiments of the present invention so as not to obscure the disclosure with details that will be readily apparent to those of ordinary skill in the art having the benefit of the description herein.

### DETAILED DESCRIPTION

Detailed descriptions of the preferred embodiment are provided herein. However, it is to be understood that the present invention may be embodied in various forms. Therefore, specific details disclosed herein are not to be interpreted as limiting but rather as a basis for the claims and as a representative basis for teaching one skilled in the art to employ the present invention in virtually any appropriately detailed system, structure, or manner.

The terminology used herein is for the purpose of describing particular embodiments only and is not intended to be limiting of the invention. As used herein, the term “and/or” includes any and all combinations of one or more of the associated listed items. As used herein, the singular forms “a,” “an,” and “the” are intended to include the plural forms as well as the singular forms unless the context clearly indicates otherwise. It will be further understood that the terms “comprises” and/or “comprising,” when used in this specification, specify the presence of stated features, steps, operations, elements, and/or components but do not preclude the presence or addition of one or more other features, steps, operations, elements, components, and/or groups thereof.

The present invention provides a novel method of a hand-held skincare tool that allows skin scrapping and massage functionality in addition to its grounding/earthing feature.

The assembly, as per its preferred embodiments, is a portable device that can be used to relax and rejuvenate the skin. The assembly can be used to massage and/or scrape skin when applying medicated oils, liniments, creams, or lotions, effectively helping promote the ingredient absorption and accelerate blood circulation for anti-aging, anti-wrinkle, and for tendon, muscle, or other tissue relief, effects.

The assembly, as per its further embodiment, provides an improved personal body grounding system for collecting and removing excess internal and extraneous electrical charges from a human body in order to return the body towards its natural electrically neutral state. The system of the present invention may also include a digital or analog personal meter capable of measuring the personal body voltage of the user.

While a specific embodiment has been shown and described, many variations are possible. With time, additional features may be employed. The particular shape or configuration of the platform or the interior configuration may be changed to suit the system or equipment with which it is used.

Having described the invention in detail, those skilled in the art will appreciate that modifications may be made to the invention without departing from its spirit. Therefore, it is not intended that the scope of the invention be limited to the specific embodiment illustrated and described. Rather, it is intended that the scope of this invention be determined by the appended claims and their equivalents.

The Abstract of the Disclosure is provided to allow the reader to quickly ascertain the nature of the technical disclosure. It is submitted with the understanding that it will not be used to interpret or limit the scope or meaning of the claims. In addition, in the foregoing Detailed Description, it can be seen that various features are grouped together in various embodiments for the purpose of streamlining the disclosure. This method of disclosure is not to be interpreted as reflecting an intention that the claimed embodiments require more features than are expressly recited in each claim. Instead, as the following claims reflect, the inventive subject matter lies in less than all features of a single disclosed embodiment. Thus, the following claims are hereby incorporated into the Detailed Description, with each claim standing on its own as a separately claimed subject matter.

1: A personal body grounding system comprising:
a grounding assembly, including a hand-held skin scraping assembly;
a ground lead having first ends conductively coupled to the skin scraping assembly; and
a second end conductively coupled to the grounding/earthing spot.
The assembly, as per claim 1, is used to provide skin care scraping and massage functionality;
The assembly, as per claim 1, is used to massage and/or scrape skin when applying medicated oils, liniments, creams, or lotions, effectively helping promote ingredient absorption and accelerate the process.
The assembly, as per claim 1, is used to provide the grounding function and help the body release excess energy through the face especially.
The assembly, as per claim 1, comes in a variety of shapes and sizes.
